(19) United States
(12) Patent Application Publication
(10) Pub. No.: US 2022/0312818 A1
(43) Pub. Date: Oct. 6, 2022

(54) PREPARATION METHOD FOR TWO-SPOTTED CRICKET POWDER OR EXTRACT THEREOF, FOOD COMPOSITION COMPRISING SAME, AND USES OF SAME

(71) Applicant: Korea University Research and Business Foundation, Seoul (KR)

(72) Inventor: Jung Soon HAN, Seoul (KR)

(73) Assignee: Korea University Research and Business Foundation, Seoul (KR)

(21) Appl. No.: 17/629,594

(22) PCT Filed: Jul. 24, 2020
(86) PCT No.: PCT/KR2020/009782

§ 371 Date: Jan. 24, 2022

(30) Foreign Application Priority Data
Jul. 25, 2019 (KR) 10-2019-0090118
Jul. 24, 2020 (KR) 10-2020-0092151

(51) Int. Cl. A23L 35/00; A23P 10/40; A23L 3/44; A23L 33/10; A61K 35/64

(52) U.S. Cl. CPC A23L 35/00; A23P 10/40; A23L 3/44; A23L 33/10; A61K 35/64

(57) ABSTRACT

The present invention relates to: Two-spotted cricket powder and an extract thereof for food, drugs, and cosmetic materials; a preparation method of the Two-spotted cricket powder; a food composition or care food prepared using the powder; and uses of the food composition prepared using the Two-spotted cricket powder for the prevention and amelioration of sarcopenia and antioxidant effects. Sarcopenia-preventive functional food prepared using Two-spotted cricket is a highly value-added novel alternative protein-source food that can make a contribution in the future to the edible insects industry and across the industries related to functional food, inner beauty food materials, and food, and is also expected to contribute to diversifying food choices and improving nutrition among older populations that are increasing in an aging society.

### TECHNICAL FIELD

The present invention relates to a method of producing a Two-spotted cricket (Gryllus bimaculatus) powder, a method of producing a Two-spotted cricket powder extract, a Two-spotted cricket powder or Two-spotted cricket powder extract produced using the production method, a composition for a food material including the same, and the use of the same as a protein source.

This application claims priority to and the benefit of Korean Patent Application No. 10-2019-0090118, filed on Jul. 25, 2019, and Korean Patent Application No. 10-2020-0092151, filed on Jul. 24, 2020, the content disclosed in the specifications and drawings of which are incorporated herein by reference in their entirety.

### BACKGROUND ART

Two-spotted cricket (Gryllus bimaculatus), a member of the order Orthoptera and family Gryllidae, is omnivorous and eats anything including grains, animal feeds, and agricultural by-products, and has a short growth period of about 90 days and does not require overwintering of eggs, allowing large-scale indoor breeding throughout the year. Two-spotted cricket is known to be diurnal during the nymph stage and nocturnal during the adult stage, and lays transparent white eggs in a soft, moist environment such as wet sand, sandy soil, sawdust, and humus. Two-spotted cricket lays eggs one by one deep in the spawning medium using a long ovipositor and lays more than 100 to 200 eggs during its lifetime, and the eggs begin to hatch after developing for about 13 days at normal breeding temperature. The eggs hatch into nymphs, which gradually change from transparent gray to dark brown, then turn completely black after a day or so. From this point on, the nymphs feed on food, develop while molting for about two months, and become adults. After the final molting, the adults survive for about a month while continuing courting behavior for mating and spawning.

Meanwhile, since a 2013 United Nations report recommended insects as food, interest in future food and the environment has increased, and research on edible insects has been actively conducted. Edible insects contain a large amount of unsaturated fatty acids, various amino acids, and nutrients such as various vitamins, minerals, and chitosan. In addition, edible insects are attracting attention as an alternative protein source which can overcome the limitations of the existing animal protein resource supply relying on livestock and secure diversity in food supply. In addition, insects emit less carbon dioxide than existing livestock during breeding, so they are an eco-friendly alternative food resource which can reduce environmental pollution.

Two-spotted cricket contains about 40 to 60% protein and about 20.0% fat, which is almost the same amount of protein and less fat than other animal protein sources, so it is very positive for modern-day people who are concerned about excessive fat intake. In addition, Two-spotted cricket contains more omega-9 fatty acids and omega-6 fatty acids than other animal protein sources and is rich in chitosan, so it is known as the breed in the world which can be bred as an edible insect and has the potential to be used in various fields related to the prevention and treatment of chronic metabolic diseases or skeletal diseases. The omega-9 fatty acids are oleic-based fatty acids which are most abundant in vegetable olive oil and are known to be effective for preventing cardiovascular disease, and the omega-6 fatty acids are essential fatty acids which have a double bond at the sixth carbon atom from the omega carbon at an end of their carbon chain and cannot be synthesized by the body, are essential nutrients for skin health by forming cell tissues, promoting body metabolism, helping body growth, and preventing dermatitis, and are known to prevent various cardiovascular diseases through lowering blood cholesterol and have anti-cancer effects. In addition, Two-spotted cricket was used as a treatment for stones and diuretics in 17th century Europe, and as a treatment for diarrhea, dysentery, and typhoid in Japan, and have been widely used as a key ingredient in immune-improving therapies and folk remedies, for example, for boosting metabolism by breaking down alcohol in the body.

In Korea, insects such as silkworm pupae, rice grasshoppers, brown mealworm larvae, Two-spotted cricket, and Bombyx Mori are officially registered as edible insects in the Korean Food Standards Codex, and mealworms, crickets, grasshoppers, and the like are mainly used in Western countries. Accordingly, considerable research and industrialization have been conducted in relation to edible insects, and it is expected that the high value-added industry related to edible insects will be further activated in the future.

However, it is not easy for general consumers to consume edible insects in the form of raw materials due to the appearance, unpleasant taste and smell of the insects and the lack of awareness of the positive aspects of edible insects. Therefore, there have been attempts to utilize useful nutrients while reducing aversion to edible insects, and in particular, nutritional components, such as unsaturated fatty acids, of edible insects and ways to improve flavors have been mainly studied. Currently, research on Two-spotted cricket is underway, and there have been attempts to use Two-spotted cricket as an aquaculture feed additive as in Korea Laid-Open Patent Application No. 10-2016-0134939 or a functional ingredient as in Korea Patent Registration No. 10-1702851, but ways to improve the convenience of intake and flavors have not been sufficiently studied, and to date, methods for preparing a powder or powder extract food material using Two-spotted cricket or using the same have not been sufficiently studied.

### DISCLOSURE

### Technical Problem

The present invention is directed to providing a method of producing a Two-spotted cricket powder and a method of producing an extract having the highest protein content from the powder.

Accordingly, the present invention is directed to providing a method of producing a Two-spotted cricket powder.

In addition, the present invention is directed to providing a Sunsik (roasted grain powder) including a Two-spotted cricket powder produced by a production method of the present invention.

In addition, the present invention is directed to providing a method of producing an extract having the highest protein content from a Two-spotted cricket powder.

In addition, the present invention is directed to providing a composition for a food material including a Two-spotted cricket extract produced by a production method of the present invention.

In addition, the present invention is directed to providing the use of the Two-spotted cricket powder or the powder extract as a food material, a health functional food material, an inner beauty food material, a pharmaceutical material, and a cosmetic material.

In addition, the present invention is directed to providing a food composition for preventing or ameliorating sarcopenia, which includes a Two-spotted cricket powder or a Two-spotted cricket extract produced by a production method of the present invention as an active ingredient.

In addition, the present invention is directed to providing an antioxidant food composition including a Two-spotted cricket powder or a Two-spotted cricket extract produced by a production method of the present invention as an active ingredient.

However, the technical objectives of the present invention are not limited to the objectives described above, and other objectives not described above can be clearly understood by those of ordinary skill in the art to which the present invention pertains from the following description.

### Technical Solution

Various aspects of the present invention provide a method of producing a Two-spotted cricket powder and a method of producing an extract having the highest protein content from the powder.

One aspect of the present invention provides a method of producing a Two-spotted cricket (Gryllus bimaculatus) powder, which includes the steps of:

(a) fasting Two-spotted cricket;

(b) steaming the fasted Two-spotted cricket; and

(c) pulverizing the steamed Two-spotted cricket with a pulverizer and thus obtaining powder.

According to one embodiment of the present invention, the production method may additionally include a step of hot air drying or freeze-drying the steamed Two-spotted cricket in step (b).

According to another embodiment of the present invention, the hot air drying may be performed at a temperature of 55 to 65° C. for 12 to 24 hours, for example, at 60° C. for 18 hours, but the present invention is not limited thereto.

According to still another embodiment of the present invention, the freeze-drying may be performed for 48 to 60 hours after rapidly freezing at a temperature of −60 to −80° C., and for example, may be performed for 50 hours after rapidly freezing at −70° C., but the present invention is not limited thereto.

According to one embodiment of the present invention, the fasting of step (a) may be performed for 24 to 72 hours, but the present invention is not limited thereto.

According to another embodiment of the present invention, the steaming of step (b) may be performed using 100 to 200° C. steam for 15 to 60 minutes, but the present invention is not limited thereto. Instead of the steaming, blanching may be performed in 100° C. boiling water for 5 to 15 minutes.

According to still another embodiment of the present invention, the pulverization of step (c) may be performed by filling the pulverizer with nitrogen so that oxidation of powder is prevented.

According to yet another embodiment of the present invention, the Two-spotted cricket powder may have antioxidant activity.

According to yet another embodiment of the present invention, the Two-spotted cricket powder may contain one or more amino acids selected from the group consisting of histidine, threonine, arginine, valine, methionine, tryptophan, phenylalanine, isoleucine, leucine, lysine, aspartic acid, glutamic acid, asparagine, serine, glutamine, glycine, alanine, taurine, γ-aminobutyric acid (GABA), tyrosine, and proline.

Another aspect of the present invention provides a Sunsik including a Two-spotted cricket powder produced by a production method of the present invention.

As used herein, the term “Sunsik (roasted grain powder)” refers to a food originating from a mixture of seven grains (brown rice, glutinous rice, barley, black bean, black sesame, perilla, and adlay), which was one of the nutrient-rich natural healthy foods which Hwarangdo of the Silla Dynasty conveniently carried because they could not cook or eat complicated meals while traveling and training all over the country. The Sunsik is a combination of various nutrients such as fiber and carbohydrates yet is light on the stomach and easy to consume, and since ancient times, its pulverized form, “Misutkaru (misu powder),” has been used in Korea as an emergency food that is convenient to carry during wartime and food for beating the heat in summer, and has been mixed with water or milk to make porridge which is used as baby food or nourishing food for the elderly. As such, Sunsik is good for all groups of people including children, the elderly, and people with a weak digestive system, helps healthy people to lose weight or take supplemental nutrition for special purposes, and is a nutritious food mainly based on vegetable protein which can be used as a meal replacement. The addition of an animal protein source derived from edible insects to a Sunsik primarily consisting of phytonutrients will make a nutritionally-enriched, balanced meal replacement.

According to one embodiment of the present invention, the Sunsik may additionally include one or more ingredients selected from the group consisting of brown rice powder, black sesame powder, almond powder, and broccoli powder, but the present invention is not limited thereto.

According to another embodiment of the present invention, the Sunsik may include 1 to 3 parts by weight of brown rice powder, 8 to 20 parts by weight of black sesame powder, 5 to 18 parts by weight of almond powder, and 10 to 40 parts by weight of broccoli powder based on 100 parts by weight of the Two-spotted cricket powder. For example, the Sunsik may include 0.5 g of brown rice powder, 4.0 g of black sesame powder, 3.0 g of almond powder, and 6.5 g of broccoli powder based on 26 g of the Two-spotted cricket powder, but the present invention is not limited thereto. The Korean Ministry of Food and Drug Safety defines high-protein food as food containing more than twice the recommended daily intake of protein per serving. Most of the Sunsik currently on the market are 40 g per serving, and since a powder containing 11 g or more of protein per serving can be labeled as a high-protein food, it is preferable to configure the Sunsik so that 40 g thereof contains 11 to 12 g (27.5 to 30%) of protein.

Still another aspect of the present invention provides a method of producing an extract having the highest protein content from a Two-spotted cricket powder, which includes the steps of:

(a) degreasing a Two-spotted cricket powder produced by a production method of the present invention; and

(b) extracting protein from the degreased Two-spotted cricket powder and thus obtaining an extract containing protein.

According to one embodiment of the present invention, the degreasing of step (a) may be performed using one or more solvents selected from the group consisting of ethanol, hexane, and petroleum ether and preferably 99.5% ethanol, but the present invention is not limited thereto.

According to another embodiment of the present invention, the degreasing of step (a) may be performed at a temperature of 35 to 45° C. for 40 to 80 minutes and may be repeated twice, but the present invention is not limited thereto.

According to still another embodiment of the present invention, the extraction of protein of step (b) may be performed using one or more solvents selected from the group consisting of sodium hydroxide, hydrochloric acid, and a carboxylic acid.

According to yet another embodiment of the present invention, the extraction of protein of step (b) may be performed at a temperature of 30 to 50° C. for 40 minutes to 80 minutes.

Yet another aspect of the present invention provides a composition for a food material, which includes a Two-spotted cricket powder produced by a production method of the present invention or a Two-spotted cricket extract produced by a production method of the present invention.

According to one embodiment of the present invention, the food material may be a food material for an inner beauty food.

According to another embodiment of the present invention, the food material may be a food material for a care food.

As used herein, the term “inner beauty food” refers to food which is also referred to as “edible cosmetics” or “beauty food” and food that improves skin health by helping various ingredients beneficial to the skin to be absorbed into the body, and individuals can choose and consume an inner beauty food in consideration of their skin condition and lifestyle, just as they choose cosmetics that are appropriate for their skin type. For example, the additional use of an inner beauty food including the above-described composition for a food material may be much more effective than when only cosmetics or drugs are used.

As used herein, the term “care food” originally means food or services designed in consideration of the elderly with weak physical strength and means foods such as meals, snacks, and precooked foods provided in nursing homes and hospitals for the elderly who have difficulties in eating and digesting food. However, as its meaning has recently been expanded to “Smile Care Food,” “care food” refers to food and health foods for various age groups, such as young adults and middle-aged people who have difficulty in eating food or have poor nutritional balance, as well as the elderly.

Yet another aspect of the present invention provides a food composition for preventing or ameliorating sarcopenia, which includes a Two-spotted cricket powder produced by a production method of the present invention or a Two-spotted cricket extract produced by a production method of the present invention as an active ingredient.

When a Two-spotted cricket powder or Two-spotted cricket extract of the present invention is used as a food additive, the Two-spotted cricket powder or Two-spotted cricket extract may be added as is or used together with other foods or food ingredients, and may be appropriately used according to a conventional method. The addition amount of the active ingredient may be appropriately determined according to the purpose of use (prevention, health care, or therapeutic treatment). In general, when producing food or beverages, the Two-spotted cricket powder or Two-spotted cricket extract of the present invention may be added in an amount of 20 to 40 wt % based on the raw material. However, when it is intended for intake for health care and hygiene or long-term intake for health promotion, the Two-spotted cricket powder or Two-spotted cricket extract of the present invention may be used in an amount less than the above range, and since it does not have any safety risk, the active ingredient may also be used in an amount exceeding the above range.

There is no particular limitation on the type of food. Examples of the food to which the above-described material can be added include meats, sausages, breads, chocolates, candies, snacks, confectioneries, pizzas, ramen noodles, other noodles, gums, dairy products such as ice cream, various soups, sauces, jellies, energy bars, beverages, teas, drinks, alcoholic beverages, hangover relievers, and vitamin complexes, and includes all health functional foods in the ordinary sense.

A health beverage composition of the present invention may include various flavoring agents, natural carbohydrates, or the like as additional ingredients, like conventional beverages. The above-described natural carbohydrates are monosaccharides such as glucose and sucrose, disaccharides such as maltose and sucrose, polysaccharides such as dextrin and cyclodextrin, and sugar alcohols such as xylitol, sorbitol, and erythritol. As a sweetener, a natural sweetener such as thaumatin or a stevia extract or a synthetic sweetener such as saccharin or aspartame may be used. The concentration of the natural carbohydrates is generally about 0.01 to 0.20 g or about 0.04 to 0.10 g per 100 mL of the composition of the present invention.

In addition, the composition of the present invention may include various nutritional supplements, vitamins, electrolytes, flavoring agents, coloring agents, pectic acid and salts thereof, alginic acid and salts thereof, organic acids, protective colloidal thickeners, pH adjusting agents, stabilizers, preservatives, glycerin, alcohols, and carbonation agents used for carbonated beverages, and the like. In addition, the composition of the present invention may include fruit flesh for preparing a natural fruit juice, a fruit juice drink, or a vegetable drink. Such ingredients may be used either alone or in combination. Although the concentration of such ingredients is not highly important, it is generally selected within the range of 0.01 to 0.20 parts by weight based on 100 parts by weight of the composition of the present invention.

Yet another aspect of the present invention provides the use of a food composition or care food including a Two-spotted cricket powder or Two-spotted cricket extract produced by a production method of the present invention for preventing or ameliorating various symptoms or diseases by ingestion.

The symptoms or diseases may include, for example, sarcopenia, which may occur in the elderly suffering from muscle loss or in patients after surgery or may occur after weight loss.

According to one embodiment of the present invention, the composition may inhibit the expression of the myostatin (MSTN) gene by suppressing the activity of an MSTN promoter.

As used herein, the term “sarcopenia” is a compound word composed of “sarco” for muscle in Greek and “penia” for loss, and means aging-associated muscle loss. Here, “muscle” means skeletal muscle and has nothing to do with smooth muscle. That is, sarcopenia usually includes a decrease in skeletal muscle distributed in the extremities and a decrease in skeletal muscle mass, which are accompanied by a decrease in muscle strength. In addition, in the case of obese people, sarcopenic obesity in which body fat is increased whereas water and muscle are decreased is common, and sarcopenic obesity may cause various musculoskeletal disorders. Therefore, in the present invention, sarcopenia is used with a meaning including sarcopenic obesity.

It is known that sarcopenia directly causes a decrease in muscle strength and, accordingly, a decrease in various body functions, disability, and an increase in the risk of death. Sarcopenia was assigned a disease code in the United States, but there is no special therapeutic agent for it, and it requires enormous capital and a lot of time and effort to develop a synthetic compound therapeutic agent, and even if a therapeutic agent is developed, it may have side effects to the human body, so its safety must be confirmed through cell testing, Institutional Review Board (IRB) approvals, and subsequent animal and human testing to be applicable to the human body. Therefore, it takes long-term efforts and capital for a therapeutic agent to go on the market. Therefore, yet another aspect of the present invention provides a food or care food for preventing or ameliorating sarcopenia, which uses Two-spotted cricket as a food material based on a natural product, can be easily ingested in everyday life, and is an eco-friendly material having no side effects.

In addition, meat and whey protein are mainly used foods for muscle enhancement or muscle function improvement, but they have problems in the supply of raw materials, environmental pollution, and side effects during long-term use. The present invention is directed to smoothly providing a food capable of enhancing muscle or improving muscle functions using Two-spotted cricket, which is an alternative protein source that does not cause environmental pollution, is easy to supply, and is rich in protein and various nutrients.

As used herein, the term “prevention” means any action which is likely to inhibit or delay the onset of sarcopenia through the ingestion of food including a Two-spotted cricket powder or Two-spotted cricket extract of the present invention as an active ingredient, and “amelioration” means any action which reduces sarcopenia-related parameters, such as the degree of reduction in muscle mass, through the ingestion of food including a Two-spotted cricket powder or Two-spotted cricket extract of the present invention as an active ingredient.

Yet another aspect of the present invention provides an antioxidant food composition including a Two-spotted cricket powder produced by a production method of the present invention or a Two-spotted cricket extract produced by a production method of the present invention as an active ingredient.

### Advantageous Effects

The present invention relates to a Two-spotted cricket powder and a food composition produced using the same, and the Two-spotted cricket powder can be used not only in the food field but also in various fields such as the functional food field, pharmaceutical field, and cosmetics field.

In addition, it is expected that the present invention can be variously used for specific target subjects (the elderly, athletes, growing and developing adolescents, people in need of weight control, patients, etc.), and specifically, it is expected that the present invention can be highly effective for treating sarcopenia in the elderly, muscle loss due to weight loss, and sarcopenic obesity, promoting the health of children and adolescents, and improving the athletic performance of athletes.

In particular, in Korea, the care food field targeting the elderly is very unfamiliar to the public, but since Korea has already entered an aging society, the present invention can provide a care food, more specifically, a Sunsik, produced for the growing elderly population by using a Two-spotted cricket powder of the present invention, which can be easily digested and absorbed even by people with weak teeth or weak gastrointestinal function as is or in the form of a porridge by adding water, milk, soymilk, or the like, and is therefore a high-quality nutritional food.

In addition, whereas a conventional Sunsik has a disadvantage in that it is made from grains and thus lacks protein and essential fatty acids, a food composition, care food, functional food, or Sunsik of the present invention produced using an edible insect, more specifically, a Two-spotted cricket powder, contains a large amount of essential amino acids, amino acids that help prevent sarcopenia, and essential fatty acids that help reduce inflammation and thus is expected to greatly contribute to preventing geriatric diseases and promoting health.

### DESCRIPTION OF DRAWINGS

FIG. 1 is a flowchart illustrating a method of producing a Two-spotted cricket extract according to the present invention.

FIGS. 2A and 2B illustrate the results of measuring the expression level of myostatin (MSTN) (FIG. 2A) and the activity of an MSTN promoter (FIG. 2B) in order to evaluate a muscle loss inhibitory effect of an extract (Cricket) and a protein extract (Cricket Protein) of a Two-spotted cricket powder of the present invention.

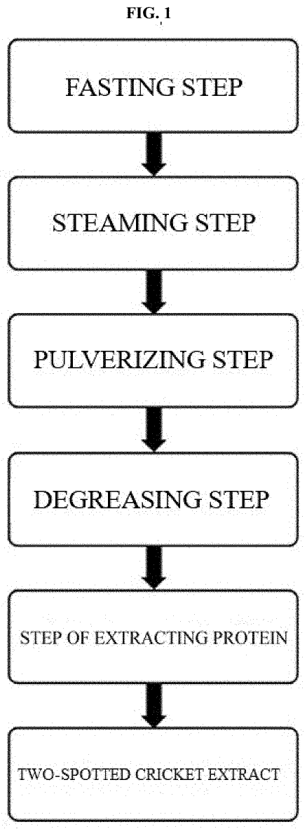

### MODES OF THE INVENTION

Terms or words used in this specification and claims should not be interpreted as being limited to commonly used meanings or meanings in dictionaries, and, based on the principle that the inventors can appropriately define concepts of terms in order to describe their invention in the best way, the terms and words should be interpreted with meanings and concepts which are consistent with the technical spirit of the present invention.

Hereinafter, exemplary embodiments are presented to help the understanding of the present invention. However, the following exemplary embodiments are only provided to facilitate understanding of the present invention, and the contents of the present invention are not limited by the exemplary embodiments.

### EXAMPLES

### Example 1

### Two-Spotted Cricket Selection Criteria for Food Materialization

Two-spotted cricket, which is the main ingredient of the present invention, was purchased from a cricket farm located in Saenggeuk-myeon, Chungcheongbuk-do (Chungcheongbuk-do, Republic of Korea). The Two-spotted cricket was fasted for two days to remove feces in the body and prevent the growth of microorganisms and thereby eliminate an unpleasant smell and improve taste.

### Example 2

### Determination of Optimal Pulverization Conditions for Food Materialization of Two-Spotted Cricket

After fasting for two days and washing, the Two-spotted cricket was pulverized through the following process. The washed Two-spotted cricket was blanched for about 10 minutes in 100° C. boiling water or steamed for about 30 minutes with 100° C. steam, and then hot air-dried at 60° C. for 18 hours or rapidly frozen at −70° C. and freeze-dried for 48 to 60 hours. The dried Two-spotted cricket was pulverized with a pulverizer to obtain a powder.

### Example 3

### Determination of Conditions for Producing Extract Having Highest Protein Content from Two-Spotted Cricket Powder

The Two-spotted cricket powder obtained by the method of Example 2 was degreased by solvent extraction with ethanol. To the obtained degreased Two-spotted cricket powder, 0.25 M sodium hydroxide (NaOH) was added in a weight ratio of 1:15 and mixed at 40° C. for 60 minutes using a hot plate (Vision Scientific Co., Ltd., Korea) and a magnetic stirrer. Subsequently, centrifugation was performed at 4° C. and 3,000 rpm for 10 minutes using a centrifuge (VS-24SMTi, Vision Science Co., Ltd., Korea), and only the supernatant was collected. After adjusting the pH of the obtained supernatant to a pH of 4.4, centrifugation was again performed at 4° C. and 3,500 rpm for 10 minutes, and thus a pellet was obtained. The pellet was put in a dialysis bag (12 kDa MWCO; Sigma-Aldrich Chemical Co., USA) and desalted for 12 hours. Subsequently, freeze-drying was performed for 36 hours, and thus a Two-spotted cricket protein extract powder was obtained. The obtained powder was maintained at a temperature of −24° C. or less for further experiments.

10 mg of the Two-spotted cricket protein extract powder was input into 1 mL of DMSO, dissolved using an ultrasonic homogenizer (Sonicator Vibra cell, Sonics & Materials; amplitude control 40%), and then centrifuged at 13,000 rpm and 10° C. for 10 minutes, and only the supernatant was collected and transferred to a 1 mL Eppendorf tube (e-tube; micro centrifuge tube).

### Example 4

### Nutritional Composition Analysis of Extract and Protein Extract of Two-Spotted Cricket Powder

4.1. Analysis of Three Major Nutrients (Carbohydrate, Fat, and Protein)

After pulverizing Two-spotted cricket, water content was analyzed by an atmospheric heating and drying method performed at 105° C., crude fat content was analyzed by a Soxhlet extraction method, and crude mineral content was analyzed by an ashing method performed at 550° C. Carbohydrate content was calculated as a value obtained by subtracting the content of water, crude protein, crude fat, and crude minerals from 100 g of sample. The crude protein content was analyzed with a nitrogen analyzer, and the analyzed nitrogen content was multiplied by a protein factor of 6.25 and expressed as the crude protein content.

The results of analyzing composition of the Two-spotted cricket powder are shown in Table 1 below. It was confirmed that the Two-spotted cricket powder contained 3.86% water, 4.41% minerals, 61.05% crude protein, 19.08% crude fat, and 11.60% carbohydrates, which shows a significantly higher protein content and lower fat content compared to other edible insects. Since the protein content is higher than the fat content, it is expected that the Two-spotted cricket powder has potential as a food material or food for preventing muscle-related diseases or muscle loss in the elderly and an increase in blood lipids in middle-aged and elderly people, and in particular, it is expected that Two-spotted cricket powder can be used as a beauty food material with potential as a weight loss material due to its low fat and carbohydrate content.

TABLE 1

| Sample | | Crude | Crude | Crude | Carbo- |
|---|---|---|---|---|---|
| (%) | Water | minerals | protein | fat | hydrates |
| Two- | 3.86 ± 0.23 | 4.41 ± 0.60 | 61.05 ± 1.06 | 19.08 ± 0.16 | 11.60 ± 0.74 |
| spotted | | | | | |
| cricket | | | | | |
| powder | | | | | |

4.2. Mineral Composition Analysis of Extract and Protein Extract of Two-Spotted Cricket Powder

To analyze mineral content, an extract and protein extract of the Two-spotted cricket powder were wet-decomposed, and the amounts of calcium (Ca), potassium (K), sodium (Na), phosphate (P), and zinc (Zn), which are minerals contained in large amounts, were measured using an optical emission spectrometer.

The results of analyzing the mineral content of the extract (cricket powder extract (CPE)) and the protein extract (cricket powder protein extract (CPPE)) of the Two-spotted cricket powder are shown in Table 2 below (units: mg/100 g). The mineral composition of the Two-spotted cricket powder extract was 110.8 mg/100 g of Ca, 724.7 mg/100 g of K, 289.3 mg/100 g of Na, 74.0 mg/100 g of P, and 22.1 mg/100 g of Zn, and the Two-spotted cricket powder protein extract contained 2.5 mg/100 g of Ca, 8.6 mg/100 g of K, 91.0 mg/100 g of Na, 98.0 mg/100 g of P, and 23.5 mg/100 g of Zn. From these results, it is expected that the Two-spotted cricket powder, which has a high Ca content, has potential for the prevention and amelioration of osteoporosis in the elderly and the promotion of health in growing children and adolescents. In addition, since K has a physiological function of lowering blood pressure, it is expected that the Two-spotted cricket powder extract will be useful as a functional material for preventing hypertension.

TABLE 2

| Sample | | | | | |
|---|---|---|---|---|---|
| (mg/kg) | Ca | K | Na | P | Zn |
| CPE | 110.8 | 724.7 | 289.3 | 74.0 | 22.1 |
| CPPE | 2.5 | 8.6 | 91.0 | 98.0 | 23.5 |

4.3. Essential Amino Acid Composition Analysis of Extract and Protein Extract of Two-Spotted Cricket Powder

The amino acid content was measured using an amino acid analyzer. In one example of a measurement method, the measurement was performed by inputting 5 g of a sample and 40 mL of 6 N HCl into a round flask and mixing, and then performing hydrolysis by injecting nitrogen gas at 110° C. for 24 hours. After concentrating HCl under reduced pressure at 50° C., the concentrated sample was diluted by adding 50 mL of 0.2 N sodium citrate buffer (pH 2.2), and filtered with 0.45 μm filter paper. After filtering, the sample (30 μL) was analyzed using an automatic amino acid analyzer, an ion change column (4.6 mm×60 mm), and a visible detector. The analysis was performed at a buffer flow rate of 1 mL/min, a column temperature of 20 to 85° C., and a reaction temperature of 50 to 140° C.

The essential amino acid compositions of the extract (cricket powder extract (CPE)) and the protein extract (cricket powder protein extract (CPPE)) of the Two-spotted cricket powder are shown in Table 3 below. The Two-spotted cricket powder extract contained essential amino acids such as 10,380.00 mg/kg of histidine, 12,679.04 mg/kg of threonine, 32,189.42 mg/kg of arginine, 17,186.31 mg/kg of valine, 6,077.52 mg/kg of methionine, 340.03 mg/kg of tryptophan, 12,249.08 mg/kg of phenylalanine, 12,790.01 mg/kg of isoleucine, 25,382.52 mg/kg of leucine, and 12,050.90 mg/kg of lysine, and the powder protein extract contained 2,763.08 mg/kg of histidine, 710.63 mg/kg of threonine, 2,822.72 mg/kg of arginine, 1,147.28 mg/kg of valine, 679.55 mg/kg of methionine, 1,367.50 mg/kg of phenylalanine, 1,281.90 mg/kg of isoleucine, 2,753.34 mg/kg of leucine, and 900.17 mg/kg of lysine. The essential amino acids are amino acids which are not synthesized in the body and thus should be ingested with food, and the Two-spotted cricket powder extract contained a large amount of each of the 10 essential amino acids. Both the Two-spotted cricket powder and the powder protein extract also contained leucine, isoleucine, and valine which are branched chain amino acids (BCAAs), and since the BCAAs are known to be oxidized in skeletal muscle and used as an energy source for the muscle, have the ability to minimize a decrease in protein in muscle cells by inhibiting protein catabolism in the muscle cells, and are used as an energy source for muscles during long-term exercise, it is expected that Two-spotted cricket, which is rich in the BCAAs, will have high utility as a food that helps to prevent muscle loss in the elderly and patients by participating in the process of muscle strength enhancement and muscle building and have high utility as a material for a nutritional supplement or health functional food not only for growing children and adolescents, but also for the general public. In addition, since Two-spotted cricket is a suitable food substitute which meets the needs of people having the risk of increased blood lipids due to excessive meat consumption and modern-day people who value health, it is expected that Two-spotted cricket will be developed as a material for food or various functional foods, and as protein supplements for athletes, nutritional supplements for patients, and new raw materials for pharmaceuticals and functional cosmetics. Two-spotted cricket also contains a large amount of lysine, and since lysine is known to help maintain skin health and suppress skin aging by participating in collagen synthesis, it is expected that Two-spotted cricket will be useful as a skin beauty-related material or a beauty food.

TABLE 3

| | Sample (mg/kg) | |
|---|---|---|
| Amino acid | CPE | CPPE |
| Histidine | 10,380.00 | 2,763.08 |
| Threonine | 12,679.04 | 710.63 |
| Arginine | 32,189.42 | 2,822.72 |
| Valine | 17,186.31 | 1,147.28 |
| Methionine | 6,077.52 | 679.55 |
| Tryptophan | 340.03 | NA |
| Phenylalanine | 12,249.08 | 1,367.50 |
| Isoleucine | 12,790.01 | 1,281.90 |
| Leucine | 25,382.52 | 2,753.34 |
| Lysine | 12,050.90 | 900.17 |

4.4. Non-Essential Amino Acid Composition Analysis of Extract and Protein Extract of Two-Spotted Cricket Powder

The non-essential amino acid compositions of the extract (cricket powder extract (CPE)) and the protein extract (cricket powder protein extract (CPPE)) of the Two-spotted cricket powder extract are shown in Table 4 below. The Two-spotted cricket powder extract contained non-essential amino acids such as 19,451.30 mg/kg of aspartic acid, 36,085.54 mg/kg of glutamic acid, 18,447.13 mg/kg of serine, 639.89 mg/kg of glutamine, 21,944.58 mg/kg of glycine, 31,947.01 mg/kg of alanine, 3,309.13 mg/kg of taurine, 17,053.70 mg/kg of tyrosine, and 2,834.81 mg/kg of proline, and the Two-spotted cricket powder protein extract contained 1,571.11 mg/kg of aspartic acid, 3,266.08 mg/kg of glutamic acid, 730.02 mg/kg of serine, 2,617.74 mg/kg of glycine, 1,965.78 mg/kg of alanine, 83.44 mg/kg of taurine, and 1,351.96 mg/kg of tyrosine. Among the non-essential amino acids, proline is involved in collagen synthesis together with lysine, so it is expected that the Two-spotted cricket powder extract or the Two-spotted cricket powder protein extract will be useful as a skin beauty-related material or a beauty food material in the future.

TABLE 4

| | Sample (mg/kg) | |
|---|---|---|
| Amino acid | CPE | CPPE |
| Aspartic acid | 19,451.30 | 1,571.11 |
| Glutamic acid | 36,085.54 | 3,266.08 |
| Asparagine | 338.44 | NA |
| Serine | 18,447.13 | 730.02 |
| Glutamine | 639.89 | NA |
| Glycine | 21,944.58 | 2,617.74 |
| Alanine | 31,947.01 | 1,965.78 |
| Taurine | 3,309.13 | 83.44 |
| γ-Aminobutyric acid (GABA) | 104.35 | NA |
| Tyrosine | 17,053.70 | 1,351.96 |
| Proline | 2,834.81 | NA |

### Example 5

### Confirmation of Antioxidant Effect of Extract and Protein Extract of Two-Spotted Cricket Powder

The antioxidant activity of the extract and the protein extract of the Two-spotted cricket powder was confirmed by measuring a total polyphenol content and DPPH radical scavenging ability. In one example of a measurement method, the total polyphenol content was measured according to a modification of the Folin-Denis method (Folin & Denis, 1912). After taking 1 mL of the diluted Two-spotted cricket powder extract and adding 1 mL of a sodium carbonate (Na₂CO₃; Sigma-Aldrich) solution thereto, the resultant was allowed to stand for three minutes and then reacted by adding 0.2 mL of a 50% Folin-Ciocalteu reagent (Sigma-Aldrich), and absorbance was measured at 750 nm using a microplate reader (BN 02514; Molecular Devices, USA). A total polyphenol content converted based on a standard curve obtained using tannic acid (Sigma-Aldrich) is shown. The DPPH radical scavenging ability of the Two-spotted cricket powder extract sample was measured according to a modification of the Blois method (1958). After adding a 1.5×10⁻⁴ M DPPH solution to 0.1 mL of the sample, the resultant was allowed to stand for 30 minutes in the dark at room temperature, and absorbance was measured at 517 nm using a microplate reader.

The results of measuring the total polyphenol content of the extract (cricket powder extract (CPE)) and the protein extract (cricket powder protein extract (CPPE)) of the Two-spotted cricket powder are shown in Table 5 below. The total polyphenol contents of the Two-spotted cricket powder extract and the powder protein extract were 6.61 mg GAE/g and 12.18 mg GAE/g, respectively, which shows that the total polyphenol content of the Two-spotted cricket powder protein extract was higher. Phenolic compounds are materials present in plants and can be used as indirect indicators of antioxidant activity, and they inhibit the generation of reactive oxygen species occurring in the body and prevent the oxidation of lipids and proteins in the body, thereby inhibiting cell damage and the abnormal growth and differentiation of cells, and thus are known as factors capable of preventing cancer by blocking the initiation stage of cancer development. Therefore, the above results suggest that the Two-spotted cricket powder protein extract having a higher total polyphenol content has better antioxidant ability and thus has potential as a material for preventing aging and various cancers.

DPPH radical scavenging ability is based on the principle that a DPPH radical, which is a stable free radical, undergoes a color change from purple (DPPH) to colorless (diphenyl-picrylhydrazine) and an absorbance change as it is reduced by sulfur-containing amino acids such as cysteine, glutathione, aromatic amines, and ascorbic acid, and is useful for measuring antioxidant ability, and the measurement results are shown in Table 5 below. The DPPH radical scavenging ability of the Two-spotted cricket powder extract and the Two-spotted cricket powder protein extract was very high, 68.25% and 80.37%, respectively, and the scavenging ability of the protein powder extract was higher. This is significantly higher than the reported DPPH radical scavenging ability of a brown mealworm ethanol extract which is 16.2%, so the Two-spotted cricket powder has a very high amino acid content and is expected to have a great value as an animal-based antioxidant food. Since antioxidants are known to suppress aging by inhibiting cellular oxidation in the body and scavenging active radicals, alleviate hypertension, and enhance various physiological functions such as immune function, it is expected that a production method of the present invention will be useful for a food material or food having excellent antioxidant activity, and furthermore, an inner beauty food.

TABLE 5

| | Sample | |
|---|---|---|
| | CPE | CPPE |
| DPPH (%) | 68.25 | 80.37 |
| Total polyphenol (mg GAE/g) | 6.61 | 12.18 |

When the results of Examples 4 and 5 are combined, it can be seen that the Two-spotted cricket powder extract and the powder protein extract contained a large amount of protein and a small amount of fat, had an excellent essential amino acid composition, and in particular, contained a large amount of valine, leucine, and isoleucine which are amino acids related to muscles. In addition, the Two-spotted cricket powder extract and the powder protein extract had high total polyphenol content and excellent DPPH radical scavenging ability. Therefore, it is expected that the Two-spotted cricket powder extract or the powder protein extract produced by a production method of the present invention will be useful as a high-quality protein food that can replace animal protein, a food material or food for promoting antioxidant activity or preventing muscle loss, and an inner beauty food.

### Example 6

### Confirmation of Sarcopenia Inhibitory Effect of Extract and Protein Extract of Two-Spotted Cricket Powder

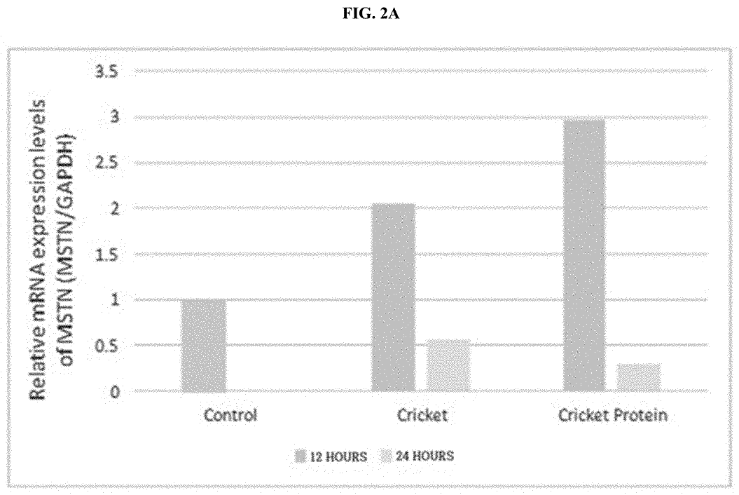

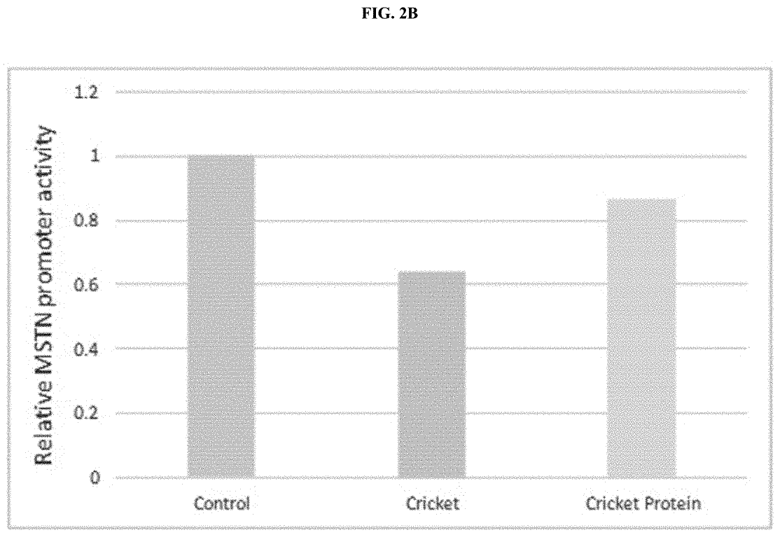

To confirm the muscle loss inhibitory effect of the extract and the protein extract of the Two-spotted cricket powder, the expression level of MSTN, which is a marker gene indicating sarcopenia, and the activity of an MSTN promoter were comparatively measured after treating the extract and the protein extract of the Two-spotted cricket powder of the present invention. The treatment of 0.01 to 1 mg/mL of the Two-spotted cricket powder extract (labeled as “Cricket” in FIGS. 2A and 2B) or the Two-spotted cricket powder protein extract (labeled as “Cricket protein” in FIGS. 2A and 2B) or less did not show toxicity to mouse skeletal muscle cells (C2C12 cell line).

Upon the treatment of 10 μg/mL of the Two-spotted cricket powder extract or the Two-spotted cricket powder protein extract for 24 hours, the mRNA expression level of MSTN gene decreased to about 77% and 44%, respectively (FIG. 2A). In addition, as a result of measuring activity using an MSTN promoter luciferase vector, it was confirmed that the Two-spotted cricket powder extract or the Two-spotted cricket powder protein extract inhibited the promoter activity by about 35.9% and 13.6%, respectively (FIG. 2B). That is, the above results prove that the Two-spotted cricket powder extract or the Two-spotted cricket powder protein extract has the effect of inhibiting sarcopenia by suppressing MSTN mRNA expression by suppressing MSTN promoter activity.

### Example 7

### Preparation of Mixed Sunsik (Roasted Grain Powder) Using Two-Spotted Cricket Powder

To the Two-spotted cricket powder of the present invention, brown rice (contains carbohydrate source, vegetable protein, fiber source, various vitamins and minerals), black sesame (contains essential fatty acids and vegetable protein), almond (contains vegetable protein and essential fatty acids), broccoli (vitamins and minerals), perilla (essential fatty acids), and the like were added to prepare a Sunsik. Since a high-protein food is defined as a food containing 11 g or more of protein, the composition ratio of the Sunsik was adjusted so that the Sunsik contained 11 g or more of protein per serving.

More specifically, the Sunsik was prepared by mixing 26 g of the Two-spotted cricket powder with 0.5 g of brown rice powder, 4.0 g of black sesame powder, 3.0 g of almond powder, and 6.5 g of broccoli powder. Since the prepared Sunsik is rich in protein, it is expected to function as a high-quality source of protein and essential fatty acids.

The above description of the present invention is for illustration, and those of ordinary skill in the art to which the present invention pertains can understand that modifications into other specific forms are possible without changing the technical spirit or essential features of the present invention. Therefore, it should be understood that the exemplary embodiments described above are illustrative in all respects and not restrictive.

### INDUSTRIAL APPLICABILITY

The present invention relates to a method of producing a Two-spotted cricket powder or an extract thereof, and a food composition and the like produced using the same. The present invention can be variously used for specific target subjects (the elderly, athletes, growing and developing adolescents, people in need of weight control, patients, etc.), and specifically, can be highly effective for treating sarcopenia in the elderly, muscle loss due to weight loss, and sarcopenic obesity, promoting the health of children and adolescents, and improving the athletic performance of athletes. In addition, a food composition, care food, functional food, or Sunsik produced using a Two-spotted cricket powder of the present invention or an extract thereof contains a large amount of essential amino acids, amino acids that help prevent sarcopenia, and essential fatty acids that help reduce inflammation and thus is expected to greatly contribute to preventing geriatric diseases and promoting health. Furthermore, the Two-spotted cricket powder or extract is expected to have high industrial applicability due to its high utility in various fields such as the food field, functional food field, pharmaceutical field, and cosmetics field.

1-22. (canceled)

23. A method of producing a Two-spotted cricket powder or extract, comprising the steps of:
(a) fasting the Two-spotted cricket;
(b) steaming the fasted Two-spotted cricket; and
(c) pulverizing the steamed Two-spotted cricket with a pulverizer and thus obtaining powder.

24. The method of claim 23, further comprising hot air drying or freeze-drying the steamed Two-spotted cricket of the step (b).

25. The method of claim 24, wherein the hot air drying is performed at a temperature of 55° C. to 65° C. for 12 hours to 24 hours; or
the freeze-drying is performed for 48 hours to 60 hours after rapidly freezing at a temperature of −60° C. to −80° C.

26. The method of claim 23, wherein the fasting of the step (a) is performed for 24 to 72 hours.

27. The method of claim 23, wherein the steaming of the step (b) is performed using 100° C. to 200° C. steam for 15 to 60 minutes.

28. The method of claim 23, wherein the pulverizing of the step (c) is performed by filling the pulverizer with nitrogen so that oxidation of powder is prevented.

29. The method of claim 23, wherein the Two-spotted cricket powder has antioxidant activity.

30. The method of claim 23, wherein the Two-spotted cricket powder contains one or more amino acids selected from the group consisting of histidine, threonine, arginine, valine, methionine, tryptophan, phenylalanine, isoleucine, leucine, lysine, aspartic acid, glutamic acid, asparagine, serine, glutamine, glycine, alanine, taurine, γ-aminobutyric acid (GABA), tyrosine, and proline.

31. The method of claim 23, further comprising the steps of:
(d) degreasing the Two-spotted cricket powder; and
(e) extracting protein from the degreased Two-spotted cricket powder and obtaining an extract containing protein.

32. The method of claim 31, wherein the degreasing of the step (d) is performed using one or more solvents selected from the group consisting of ethanol, hexane, and petroleum ether; or
the degreasing of the step (d) is performed at a temperature of 35° C. to 45° C. for 40 minutes to 80 minutes.

33. The method of claim 31, wherein the extracting of protein of the step (e) is performed using one or more solvents selected from the group consisting of sodium hydroxide, hydrochloric acid, and a carboxylic acid; or
the extracting of protein of the step (e) is performed at a temperature of 30° C. to 50° C. for 40 minutes to 80 minutes.

34. A composition for a food material, comprising:
the Two-spotted cricket powder or extract produced by the method of claim 23.

35. The composition of claim 34, wherein the food material is a food material for an inner beauty food.

36. The composition of claim 34, wherein the composition has antioxidant activity.

37. The composition of claim 34, wherein the food material is for a Sunsik (roasted grain powder).

38. The composition of claim 37, wherein the Sunsik (roasted grain powder) further comprises one or more ingredients selected from the group consisting of brown rice powder, black sesame powder, almond powder, and broccoli powder.

39. The composition of claim 37, wherein the Sunsik (roasted grain powder) includes 1 part by weight to 3 parts by weight of brown rice powder, 8 parts by weight to 20 parts by weight of black sesame powder, 5 parts by weight to 18 parts by weight of almond powder, or 10 parts by weight to 40 parts by weight of broccoli powder based on 100 parts by weight of the Two-spotted cricket powder.

40. A method for ameliorating or treating sarcopenia, comprising:
administering a composition comprising the Two-spotted cricket powder or extract produced by the method of claim 23 as an active ingredient, into a subject in need thereof.

41. The method of claim 40, wherein the powder or extract inhibits expression of a myostatin (MSTN) gene by suppressing activity of an MSTN promoter.

42. The method of claim 40, wherein the composition has antioxidant activity.
